(19) United States
(12) Patent Application Publication
(10) Pub. No.: US 2019/0250155 A1
(43) Pub. Date: Aug. 15, 2019

(54) BEAD-BASED ENZYMATIC ASSAY

(71) Applicants: Abhinav Bhushan, Chicago, IL (US); Sonali Karnik, Indianapolis, IN (US)

(72) Inventors: Abhinav Bhushan, Chicago, IL (US); Sonali Karnik, Indianapolis, IN (US)

(73) Assignee: ILLINOIS INSTITUTE OF TECHNOLOGY, CHICAGO, IL (US)

(21) Appl. No.: 16/270,387

(22) Filed: Feb. 7, 2019

Related U.S. Application Data
(60) Provisional application No. 62/627,381, filed on Feb. 7, 2018.

(51) Int. Cl. G01N 33/543; B01L 3/00; G01N 33/533; G01N 33/68; G01N 33/92

(52) U.S. Cl. CPC G01N 33/54313; B01L 3/502761; B01L 3/502715; G01N 33/533; C12Y 301/01013; G01N 33/92; C12Y 207/0103; C12Y 101/03021; G01N 33/6803

(57) ABSTRACT

An apparatus and method of detecting components in a microfluidic sample. The sample and a plurality of microbeads are mixed within the microfluidic device. Each of the microbeads comprises a plurality of bioactive proteins bound thereon. A fluorescent signal is generated from a reaction of the microbeads and the microfluidic sample. The generated fluorescent signal is then able to be detected, wherein an intensity of fluorescence is directly proportional to a concentration of the peroxide generated.

### CROSS REFERENCE TO RELATED APPLICATION

This application claims the benefit of U.S. Provisional Application, Ser. No. 62/627,381, filed on 7 Feb. 2018. The co-pending Provisional Application is hereby incorporated by reference herein in its entirety and is made a part hereof, including but not limited to those portions which specifically appear hereinafter.

### GOVERNMENT SUPPORT CLAUSE

This invention was made with government support under contract 7R00DK095984-04 awarded by the National Institutes of Health. The government has certain rights in the invention.

### FIELD OF THE INVENTION

This invention relates generally to detection assays and, more particularly, to a device and method incorporating fluidics, bead-based enzymatic assays, and/or fluorescence for target detection, such as cholesterol detection.

### BACKGROUND OF THE INVENTION

Microfluidics based tissue engineering mimics the physiological environment of tissues and organs. Perfusion is one process mimicked by microfluidics; however, continuous measurement of cellular secretion requires the analysis to be on-line. One approach for on-line analysis includes enzymatic assay on-a-chip using mixing channels for reagents and sample

One of the benefits of the microfluidic tissue systems is that the cells can be subjected to physiological shear stress by perfusion. However, this brings upon a challenge in determining cellular function because on-line or continuous analysis of secretory components in very limited. Moreover, these secretions are generated in low volumes which further makes it challenging for analysis. There is a continuing need for improved microfluidic-based assays.

### SUMMARY OF THE INVENTION

A general object of the invention is to provide an improved detection assay. The general object of the invention can be attained, at least in part, through a method of and apparatus for detecting components in a microfluidic sample.

The method of embodiments of this invention include: providing the microfluidic sample in a microfluidic device; introducing a plurality of microbeads to the sample within the microfluidic device, wherein each of the microbeads comprises a plurality of bioactive proteins, molecules, lipoproteins, or combinations thereof, bound thereon; generating a fluorescent signal from a reaction of the microbeads and the microfluidic sample; and detecting or measuring the generated fluorescent signal.

In embodiments of this invention the microfluidic device comprises mixing channels. In embodiments of this invention the microbeads comprise polystyrene beads. In embodiments of this invention the bioactive protein comprises streptavidin or an enzyme.

In embodiments of this invention the method includes generating a fluorescent signal by generating peroxide, wherein an intensity of fluorescence is directly proportional to a concentration of the peroxide generated. In embodiments of this invention generating a fluorescent signal includes oxidizing a fluorogenic material. Desirably the signal is detected and measured in real-time or near real-time. In embodiments of this invention the signal is detected intermittently or continuously for a determined period of time, without replacing or replenishing reagents.

Other objects and advantages will be apparent to those skilled in the art from the following detailed description taken in conjunction with the appended claims and drawings.

### BRIEF DESCRIPTION OF THE DRAWINGS

FIG. 1 illustrates an assay principle with chemical reactions taking place to generate a fluorescent signal according to one embodiment of this invention.

FIG. 2 illustrates an assay principle with chemical reactions taking place to generate a fluorescent signal according to one embodiment of this invention.

FIG. 3 is a schematic representation of a microfluidic culture of liver cells and detection device with the fluorescent bead used for analysis, according to one embodiment of this invention.

FIG. 4 is a schematic representation of a microfluidic detection device with fluorescent beads near an imaging port, according to one embodiment of this invention.

FIG. 5 is a correlation plot showing comparison between conventional and bead-based enzymatic assay for hydrogen peroxide.

FIG. 6 is a correlation plot showing comparison between conventional and bead-based enzymatic assay for cholesterol.

FIG. 7 is a plot of cholesterol quantified by a bead-based enzymatic assay from the perfusion based microfluidic hepatocyte cultures.

### DETAILED DESCRIPTION OF THE INVENTION

A general object of the invention is to provide an assay system or method, such as an enzymatic assay, to detect components in the low volume of microfluidic samples. Microfluidics enables precise engineering of microphysiological environments for in vitro mimicking of tissues and organs. These ‘organs-on-a-chip’ beneficially use lower number of cells and lower quantities of reagents than conventional processes.

Embodiments of this invention include a bead-based enzymatic assay ‘on-a-chip’. The use of beads (e.g., 20 nm to 200 μm in diameter) for the assay minimizes the assay volume and addresses the problem of the conventional assays requiring high volumes of samples and reagents. The invention provides an assay that is simpler than current techniques and is very versatile to be used in all enzyme based assays, such as for glucose, glycerol, triglyceride, and/or cholesterol. In embodiments of the invention, the polystyrene bead is bound with streptavidin, and used to immobilize the enzyme via biotin.

The enzymatic assay of this invention can incorporate detection by fluorescence or other suitable detection means. The invention measures target composition levels by reading the fluorescent signal generated by an analyte and the enzymes using laser excitation and a photomultiplier tube. In bead-based assays, standard fluorescent microscopy can be used to capture the individual fluorescent beads. The images can be analyzed, such as using ImageJ, to quantify the target, such as cholesterol secreted by the cells.

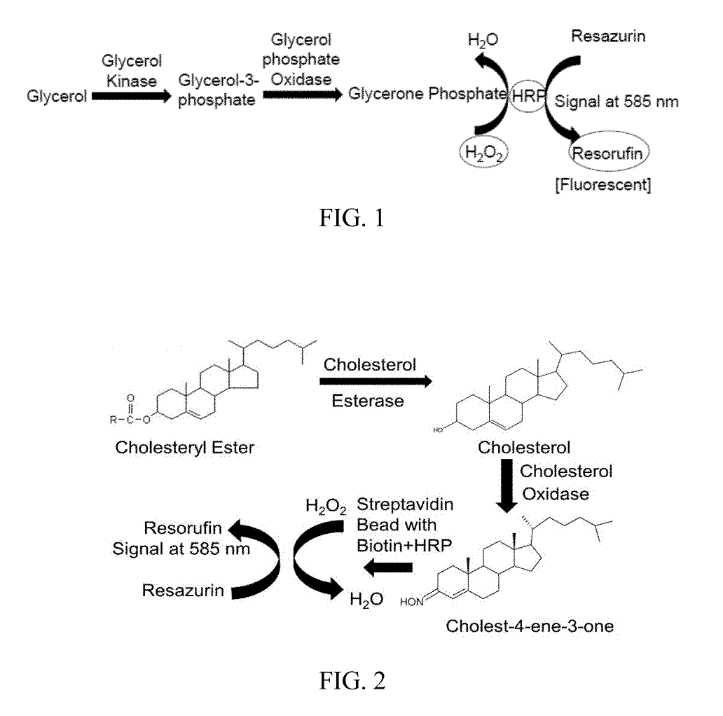

Embodiments of this invention include a bead-based enzymatic assay with horseradish peroxidase (HRP) enzyme immobilized on the polystyrene beads. As shown in FIGS. 1 and 2, the HRP enzyme catalyzes the reaction of oxidizing non-fluorescent resazurin to fluorescent resorufin in the presence of hydrogen peroxide which is generated during reaction between different substrates such as cholesterol, glucose, glycerol, pyruvate, etc. and oxidase enzymes. The oxidation reaction of resazurin to resorufin in the presence of hydrogen peroxide takes place on the beads making the beads fluorescent. The intensity of fluorescence is directly proportional to the concentration of hydrogen peroxide generated during the reaction. The fluorescent signal can be detected by a microplate reader, fluorescence microscope, or flow cytometry depending on the application. The bead-based enzymatic assay has been used to quantify, for example, hydrogen peroxide and cholesterol using both a microplate reader and fluorescence microscopy for detection.

The invention provides benefits over conventional solution based enzymatic assays, in particular because: the conventional assays use a microplate reader for detection of signal and these cannot be adapted to other analytical formats such as fluorescence microscopy or flow cytometry, and continuous real-time monitoring of biomolecules such as cholesterol is not possible for cell based or biological applications using conventional assays, typically because the medium or the sample needs to be collected before running the assay.

Bead-based enzymatic assays provide the fluorescent signal localized on a solid substrate, e.g., a polystyrene bead, making the assay adaptable to different formats such as fluorescence microscopy, flow cytometry, and microplate reader. It is possible to monitor secretions of biomolecules such as cholesterol in real-time using continuous collection of samples or medium and running the assay. For example, samples from perfusion based microfluidic hepatocyte cultures or liver-on-a-chip can be used with the bead-based enzymatic assay for real-time monitoring of cholesterol.

Oxidation reaction involving HRP generates the fluorescent signal on the bead as HRP is immobilized on the bead. Visual and image analysis is possible because of localized fluorescent signal. Real-time continuous monitoring of biomolecules secreted by the perfusion based microfluidic cultures and organs-on-a-chip is possible as the fluorescent signal on the beads can be detected and quantified using fluorescent microscopy and image analysis. Unlike the conventional enzymatic assays which are suitable for detection by microplate readers only, the bead-based enzymatic assay can be used in microplate readers, and also with fluorescence microscopy as well as flow cytometry.

The system and method of this invention are useful for detection of biomolecules such as hydrogen peroxide, cholesterol, glycerol, glucose, pyruvate, or any such molecules which use the oxidation reaction of resazurin to resorufin catalyzed by HRP for signal generation in biological samples using detection methods such as microplate reader, fluorescence microscopy, or flow cytometry. The system and method can be used to study cellular or biological response in the form of secretion of biomolecules such as hydrogen peroxide, cholesterol, glycerol, glucose, pyruvate, etc., to different biochemical stimuli using microplate readers or fluorescence microscope for detecting the signal. The system and method can be used in cytometric bead array format in flow cytometry for assessing the enzymatic or oxidative activity

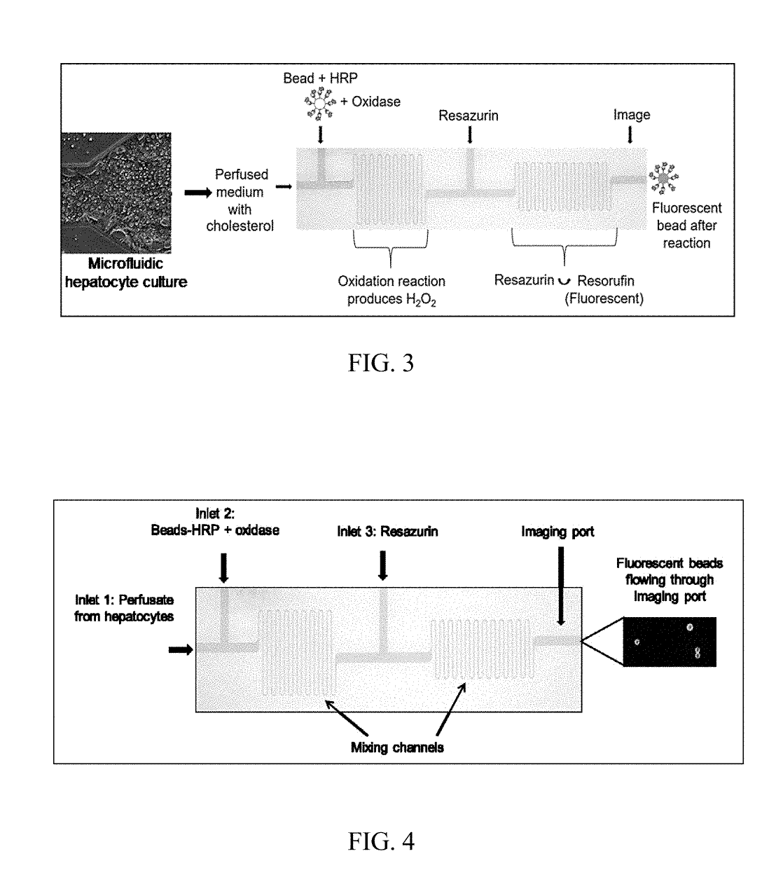

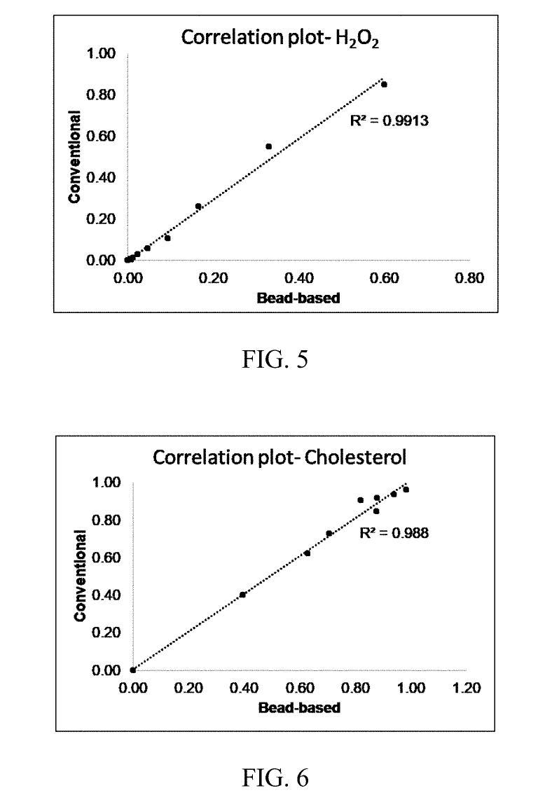

The bead-based enzymatic assay for the chemistry of oxidation reaction of resazurin to resorufin catalyzed by HRP was tested using the enzyme immobilized beads and hydrogen peroxide standards. FIGS. 3 and 4 illustrate experimental setups and assay design in microfluidic devices, wherein HRP plus biotin was immobilized on polystyrene assay beads with streptavidin. The assay was carried out in a microplate reader and compared against a commercially available assay to detect hydrogen peroxide and the results were comparable (FIG. 5). Then the assay was tested with cholesterol standards and compared with a commercially available assay for cholesterol. The detection was carried out in a microplate reader and the results were comparable (FIG. 6).

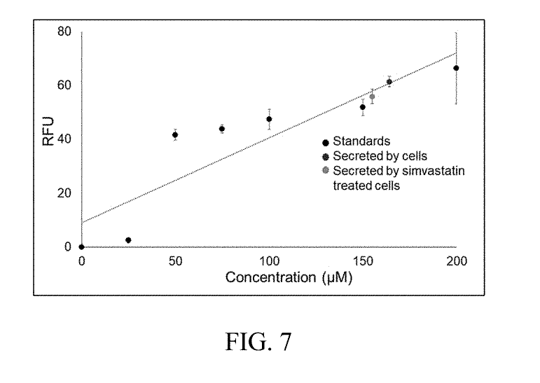

The assay has also been used to measure cholesterol secreted from liver hepatoma cells that are cultured on a microfluidic device (FIG. 3). FIG. 7 shows the cholesterol quantified by the bead-based enzymatic assay from the perfusion based microfluidic hepatocyte cultures. The enzymatic cholesterol assay retained its sensitivity after immobilizing the enzyme on the beads and reported the cellular secretion in real-time.

Thus, the invention provides an enzymatic assay to detect components in low volume of microfluidic samples. The bead-based enzymatic assay on-a-chip can be used to detect cholesterol in low volume of microfluidic samples.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method of detecting components in a microfluidic sample, comprising:
providing the microfluidic sample in a microfluidic device;
introducing a plurality of microbeads to the sample within the microfluidic device, wherein each of the microbeads comprises a plurality of bioactive proteins, molecules, lipoproteins, or combinations thereof, bound thereon;
generating a fluorescent signal from a reaction of the microbeads and the microfluidic sample; and
detecting or measuring the generated fluorescent signal.

2. The method of claim 1, wherein the microfluidic device comprises mixing channels.

3. The method of claim 1, wherein the microbeads comprise polystyrene beads.

4. The method of claim 1, wherein the bioactive protein comprises streptavidin or an enzyme.

5. The method of claim 1, wherein generating a fluorescent signal comprises generating peroxide, wherein an intensity of fluorescence is directly proportional to a concentration of the peroxide generated.

6. The method of claim 1, wherein generating a fluorescent signal comprises oxidizing a fluorogenic material.

7. The method of claim 1, wherein the signal is detected and measured in near real-time.

8. The method of claim 1, wherein the signal is detected intermittently or continuously for a determined period of time, without replacing or replenishing reagents.
